(19) United States
(12) Patent Application Publication
(10) Pub. No.: US 2026/0165735 A1
(43) Pub. Date: Jun. 18, 2026

(54) Systems and Methods for Tracking Medical Devices

(71) Applicant: Bard Access Systems, Inc., Salt Lake City, UT (US)

(72) Inventors: Jeanette E. Southard, Castleconnell (IE); Matthew J. Prince, Herriman, UT (US); Tab Robbins, Layton, UT (US); Tyler L. Durfee, Stansbury, UT (US)

(21) Appl. No.: 19/534,700

(22) Filed: Feb. 9, 2026

Related U.S. Application Data
(63) Continuation of application No. 16/777,685, filed on Jan. 30, 2020, now Pat. No. 12,544,101.
(60) Provisional application No. 62/798,930, filed on Jan. 30, 2019.

(51) Int. Cl. A61B 17/34; A61B 8/00; A61B 8/08; A61B 17/00; A61B 34/20; A61M 25/01

(52) U.S. Cl. CPC A61B 17/3403; A61B 8/085; A61B 8/4444; A61B 8/461; A61B 8/5223; A61B 2017/00106; A61B 2017/00115; A61B 2017/3413; A61B 2034/2051; A61M 25/0108

(57) ABSTRACT

Systems and methods for tracking medical devices such as needles and catheters. For example, an ultrasound-imaging system is configured to perform a set of operations for accessing a blood vessel, recommending a proper approach angle for approaching the blood vessel with the medical device, recommending a proper insertion angle for inserting the medical device in the blood vessel, ensuring a final placement of a sufficient length of the medical device within the blood vessel, and following, or tracking, a procedure for placing the medical device in the blood vessel. In addition, the ultrasound-imaging system is configured to perform a set of operations for optimizing an ultrasound image about the blood vessel or a targeted location of the blood vessel.

### PRIORITY

This application is a continuation of U.S. patent application Ser. No. 16/777,685, filed Jan. 30, 2020, now U.S. Pat. No. 12,544,101, which claims the benefit of priority to U.S. Provisional Application No. 62/798,930, filed Jan. 30, 2019, each of which is incorporated by reference in its entirety into this application.

### BACKGROUND

Medical device tracking is described for various instruments, such as catheters, stylets, and needles, in the following U.S. patents and publications, each of which is incorporated by reference in its entirety into this application: U.S. Pat. Nos. 9,554,716; 9,456,766; 9,492,097; 10,524,691; and 10,449,330.

### SUMMARY

Disclosed herein is a non-transitory computer-readable medium (“CRM”) including executable instructions that cause an ultrasound-imaging system to perform a set of operations for accessing a blood vessel when the instructions are executed by one or more processors of the ultrasound-imaging system, the set of operations including, in some embodiments determining a depth of the blood vessel with vessel depth-determination logic using ultrasound-probe data gathered above the blood vessel as input; calculating whether a medical device is able to access the blood vessel with medical device-accessibility logic using the depth of the blood vessel, an effective length of the medical device, an insertion location above the blood vessel, and an insertion angle of the medical device as inputs; and displaying a visual indicator on a display over an ultrasound image, emitting an audio indicator from a speaker, or both to indicate whether the medical device will access the blood vessel.

In some embodiments, the set of operations further includes determining the insertion location and the insertion angle of the medical device from sensor readings from a plurality of medical-device sensors of the ultrasound probe.

In some embodiments, the visual indicator is a target overlying the ultrasound image on the display. The target fades away, vanishes, switches from one color to another color, or switches from one pattern to another pattern to indicate the medical device is not able to access the blood vessel.

In some embodiments, the visual indicator is an elongate graphical element overlying the ultrasound image that represents the effective length of the medical device.

Also disclosed herein is a non-transitory CRM including executable instructions that cause an ultrasound-imaging system to perform a set of operations for ensuring a final placement of a sufficient length of a medical device within a blood vessel when the instructions are executed by one or more processors of the ultrasound-imaging system, the set of operations including, in some embodiments, determining a depth of the blood vessel with vessel depth-determination logic using ultrasound-probe data gathered above the blood vessel as input; calculating whether a minimum length of the medical device is or will be placed within the blood vessel with medical device-placement logic from the depth of the blood vessel, an effective length of the medical device, an insertion location above the blood vessel, and an angle of approach of the medical device as inputs; displaying a visual indicator on a display over an ultrasound image, emitting an audio indicator from a speaker, or both to indicate whether a potential placement of the medical will result in the final placement of a sufficient length of the medical device within the blood vessel.

In some embodiments, the set of operations further includes determining the insertion location and the angle of approach of the medical device from sensor readings from a plurality of medical-device sensors of the ultrasound probe.

In some embodiments, the visual indicator is a target overlying the ultrasound image on the display and the target fades away, vanishes, switches from one pattern to another pattern, or switches from one color to another color to indicate the potential placement of the medical device will not result in the final placement of a sufficient length of the medical device within the blood vessel.

In some embodiments, the minimum length of the medical device is user defined or set in accordance with a known minimum length provided by a manufacturer of the medical device.

In some embodiments, the medical device is a needle.

In some embodiments, the medical device is a short-length catheter.

In some embodiments, the set of operations further includes estimating a distance a tip of the catheter is from a tip of a needle in the blood vessel with tip-estimation logic as the catheter is advanced over the needle; and displaying an estimation of the distance on the display over the ultrasound image.

Also disclosed herein is a CRM including executable instructions that cause an ultrasound-imaging system to perform a set of operations for recommending a proper approach angle for approaching a blood vessel with a medical device when the instructions are executed by one or more processors of the ultrasound-imaging system, the set of operations including, in some embodiments, determining a presence of the medical device from sensor readings from a plurality of medical-device sensors of an ultrasound probe; and displaying a visual indicator on a display over an ultrasound image to indicate the proper approach angle for approaching the blood vessel with the medical device.

In some embodiments, the set of operations further includes determining a trajectory of the medical device with trajectory-determination logic using the sensor readings as input; and displaying the trajectory of the medical device over the ultrasound image on the display. The visual indicator is incorporated into the trajectory of the medical device on the display.

In some embodiments, the set of operations further includes issuing a visible warning on the display over the ultrasound image or an audible warning from a speaker if the trajectory is determined to pass through an artery.

In some embodiments, the visual indicator switches from one pattern to another pattern, one color to another color, or from a dashed line to a solid line to indicate the trajectory of the medical device follows the proper approach angle for approaching the blood vessel with the medical device.

In some embodiments, the proper approach angle is set in accordance with a recommendation by a manufacturer of the medical device, an established medical procedure for the medical device, or a user's preference for using the medical device.

Also disclosed herein is a non-transitory CRM including executable instructions that cause an ultrasound-imaging system to perform a set of operations for recommending a proper insertion angle for inserting a medical device in a blood vessel when the instructions are executed by one or more processors of the ultrasound-imaging system, the set of operations including, in some embodiments, determining a presence of the medical device from sensor readings from a plurality of medical-device sensors of an ultrasound probe; and displaying a visual indicator on a display over an ultrasound image to indicate the proper insertion angle for inserting the medical device in the blood vessel.

In some embodiments, the visual indicator appears over the ultrasound image on the display at a time the medical device reaches the blood vessel but before insertion of the medical device in the blood vessel.

In some embodiments, the visual indicator is continuously shown over the ultrasound image on the display with two or more differently colored or patterned zones. One zone of the two or more zones is enhanced to indicate whether an approach of the medical device is in accordance with the proper insertion angle for inserting the medical device in the blood vessel.

In some embodiments, the proper insertion angle is set in accordance with a recommendation by a manufacturer of the medical device, an established medical procedure for the medical device, or a user's preference for using the medical device.

Also disclosed herein is a non-transitory CRM including executable instructions that cause an ultrasound-imaging system to perform a set of operations for optimizing an ultrasound image about a blood vessel or a targeted location of the blood vessel when the instructions are executed by one or more processors of the ultrasound-imaging system, the set of operations including, in some embodiments, detecting the blood vessel using ultrasound signals echoed off the blood vessel and received by an ultrasound probe; and adjusting one or more ultrasound-probe parameters selected from a focus of the ultrasound probe, an operating frequency of the ultrasound probe, and an acoustic power output of the ultrasound probe above the blood vessel or the targeted location of the blood vessel, thereby optimizing the ultrasound image about the blood vessel or the targeted location of the blood vessel.

In some embodiments, the set of operations further includes determining the targeted location from a hysteretic analysis of ultrasound-probe locations above the blood vessel.

In some embodiments, the set of operations further including determining with blood vessel-occupation logic a percentage of the blood vessel to be occupied by a medical device upon insertion of a sufficient length of the medical device in the blood vessel.

Also disclosed herein is a non-transitory CRM including executable instructions that cause an ultrasound-imaging system to perform a set of operations for following a procedure for placing a medical device in a blood vessel when the instructions are executed by one or more processors of the ultrasound-imaging system, the set of operations including, in some embodiments, tracking a location of a tip of the medical device from a time of insertion at an insertion location, through a period of access in a targeted location of the blood vessel, to a time of withdrawing the tip of the medical device from the insertion location. The tracking includes recording a duration of the procedure including intervals thereof, a depth of the blood vessel, an angle of approach to the blood vessel, an insertion angle at the targeted location of the blood vessel, a number of readjustment passes during the procedure, or a combination thereof.

These and other features of the concepts provided herein will become more apparent to those of skill in the art in view of the accompanying drawings and following description, which disclose particular embodiments of such concepts in greater detail.

### BRIEF DESCRIPTION OF DRAWINGS

FIG. 1 illustrates a patient and an ultrasound-imaging system for placing needles and other medical devices in accordance with some embodiments.

FIG. 2 illustrates a block diagram depicting various elements of the ultrasound-imaging system in accordance with some embodiments.

FIG. 3 illustrates an ultrasound probe of the ultrasound-imaging system of FIGS. 1 and 2 in accordance with some embodiments.

FIG. 4 illustrates a needle configured for use with the ultrasound-imaging system of FIGS. 1 and 2 in accordance with some embodiments.

FIG. 5 illustrated an end-on view of the needle of FIG. 4 in accordance with some embodiments.

FIG. 6 illustrates a first view of the ultrasound probe of the ultrasound-imaging system being used to guide percutaneous insertion of a needle into a patient in accordance with some embodiments.

FIG. 7 illustrates a second view of the ultrasound probe of the ultrasound-imaging system being used to guide the percutaneous insertion of a needle into a patient in accordance with some embodiments.

FIG. 8 illustrates a simplified version of a first screenshot from a display of the ultrasound-imaging system showing a position and orientation of a needle according in accordance with some embodiments.

FIG. 9 illustrates a simplified version of a second screenshot from the display of the ultrasound-imaging system showing a position and orientation of a needle according in accordance with some embodiments.

FIG. 10 illustrates a simplified version of a third screenshot from the display of the ultrasound-imaging system showing a position and orientation of a needle according in accordance with some embodiments.

FIG. 11 illustrates a view of the ultrasound probe of the ultrasound-imaging system being used to guide insertion of a combination of a catheter and a needle into a blood vessel of a patient in accordance with some embodiments.

FIG. 12 illustrates a simplified version of a screenshot from the display of the ultrasound-imaging system graphically showing an angle of approach guide for insertion of the combination of the catheter and the needle into the blood vessel of the patient in accordance with some embodiments.

FIG. 13 illustrates a view of the ultrasound probe of the ultrasound-imaging system being used to guide threading of a catheter off a needle into a blood vessel of a patient in accordance with some embodiments.

FIG. 14 illustrates a view of the ultrasound probe of the ultrasound-imaging system being used to calculate an amount of a catheter in a blood vessel of a patient in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

With respect to “logic” or “engine,” logic and engine are independently representative of hardware, firmware, software, or a combination thereof configured to perform one or more functions. As hardware, the logic (or engine) can include circuitry having data processing, storage functionality, or a combination thereof. Examples of such circuitry includes, but are not limited or restricted to a processor, a programmable gate array, a microcontroller, an application specific integrated circuit, wireless receiver, transmitter or transceiver circuitry, semiconductor memory, or combinatorial logic.

Alternatively, or in combination with the foregoing circuitry, the logic (or engine) can be software in the form of one or more software modules, which can be configured to operate as its counterpart circuitry. The software modules can include an executable application, a daemon application, an application programming interface (“API”), a subroutine, a function, a procedure, an applet, a servlet, a routine, source code, a shared library or dynamic load library, or even one or more instructions. The software module(s) can be stored in any type of a suitable non-transitory storage medium, or transitory storage medium (e.g., electrical, optical, acoustical or other form of propagated signals such as carrier waves, infrared signals, or digital signals). Examples of non-transitory storage medium include, but are not limited or restricted to, a programmable circuit; a semiconductor memory; non-persistent storage such as volatile memory (e.g., any type of random access memory [“RAM”]); persistent storage such as non-volatile memory (e.g., read-only memory [“ROM”], power-backed RAM, flash memory, phase-change memory, etc.), a solid-state drive, hard disk drive, an optical disc drive, or a portable memory device. As firmware, the logic (or engine) can be stored in persistent storage.

With respect to “computerized” such as in a “computerized method,” computerized generally represents any corresponding operations are conducted by hardware in combination with software or firmware of a system.

Unless defined otherwise, all technical and scientific terms used herein have the same meaning as commonly understood by those of ordinary skill in the art.

Disclosed herein are systems and methods for tracking medical devices, such as needles and catheters. For example, an ultrasound-imaging system is configured to perform a set of operations for accessing a blood vessel, recommending a proper approach angle for approaching the blood vessel with the medical device, recommending a proper insertion angle for inserting the medical device in the blood vessel, ensuring a final placement of a sufficient length of the medical device within the blood vessel, and following, or tracking, a procedure for placing the medical device in the blood vessel. In addition, the ultrasound-imaging system is configured to perform a set of operations for optimizing an ultrasound image about the blood vessel or a targeted location of the blood vessel.

### Systems

Various embodiments described herein are generally directed to an ultrasound-imaging system configured to locate and guide a needle or another medical device (e.g., catheters) during ultrasound-based or other suitable procedures for accessing with the needle a subcutaneous blood vessel of a patient, for instance. In some embodiments, the system enables the position, orientation, and advancement of the needle to be superimposed in real-time atop the ultrasound image of the blood vessel, thus enabling a clinician to accurately guide the needle to the intended target. Furthermore, in some embodiments, the system tracks the needle's position in five degrees of motion: x, y, and z spatial coordinate space, needle pitch, and needle yaw. Such tracking enables the needle to be guided and placed with relatively high accuracy.

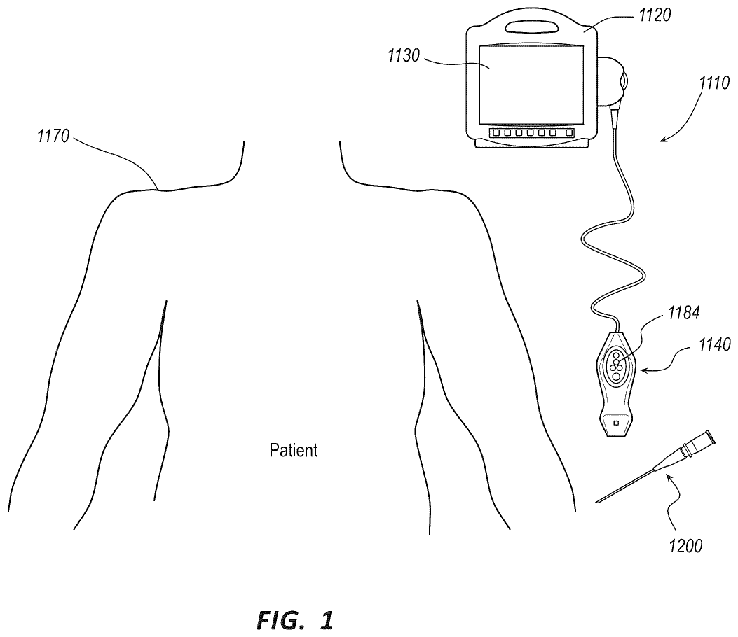

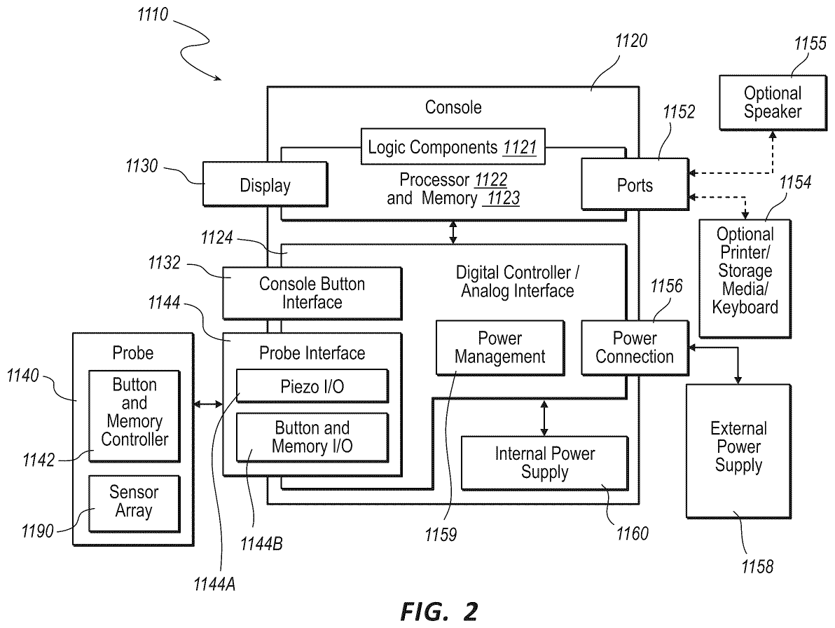

Reference is first made to FIGS. 1 and 2, which depict various components of the ultrasound-imaging system, generally designated as 1110, configured in accordance with some embodiments. As shown, the ultrasound-imaging system 1110 generally includes an ultrasound imaging portion including a console 1120, display 1130, and probe 1140, each of which is described in further detail below. It should be noted, however, that the ultrasound imaging portion can be configured in any of a variety of ways in addition to what is shown and described herein. The ultrasound-imaging portion of the ultrasound-imaging system 1110 is employed to image a targeted internal portion of a body of a patient prior to percutaneous insertion of a needle or other device to access the target. As described below, in some embodiments insertion of the needle is performed prior to the subsequent insertion of a catheter into a vein or other portion of the vasculature of the patient. It is appreciated, however, that insertion of a needle into the body of a patient can be performed for a variety of medical purposes.

FIG. 1 shows the general relation of the above-described components to a patient 1170 during a procedure to, for example, place a catheter into the patient's vasculature through a skin insertion site in accordance with some embodiments. Such a catheter generally includes a proximal portion that remains exterior to the patient and a distal portion that resides within the patient vasculature after placement is complete. The ultrasound-imaging system 1110 is employed in some embodiments to ultimately position a distal tip of the catheter in a desired position within the patient's vasculature. In some embodiments, the desired position for the distal tip of the catheter is proximate the patient's heart, such as in the lower one-third portion of the superior vena cava (“SVC”). Of course, the ultrasound-imaging system 1110 can be employed to place the distal tip in other locations.

The proximal portion of the catheter further includes a hub that provides fluid communication between one or more lumens of the catheter and one or more extension legs extending proximally from the hub. As mentioned, placement of a needle into the patient's vasculature at the skin insertion site is typically performed prior to insertion of the catheter, though it is appreciated that other placement methods can be employed such as simultaneously placing a combination of the needle and catheter into the patient's vasculature. Further, it is appreciated that the ultrasound-imaging system 1110 can be employed for a variety of additional uses such as needle insertion for insertion of other medical devices into the body of a patient including X-ray or ultrasound markers, biopsy sheaths, ablation components, bladder scanning components, vena cava filters, etc.

In greater detail, the console 1120 houses a variety of components of the ultrasound-imaging system 1110 and it is appreciated that the console 1120 can take one of a variety of forms. A processor 1122, including non-volatile memory 1123 such as electrically erasable programmable read-only memory (“EEPROM”) for instance, is included in the console 1120 for controlling system functions and operating various logic components 1121 during operation of the ultrasound-imaging system 1110, thus acting as a control processor. The logic components 1121 include, but are not limited to, vessel depth-determination logic, medical device-accessibility logic, medical device-placement logic, tip-estimation logic, trajectory-determination logic, and blood vessel-occupation logic, which logic uses various inputs as set forth herein. A digital controller/analog interface 1124 is also included with the console 1120 and is in communication with both the processor 1122 and other system components to govern interfacing between the probe 1140 and other ultrasound-imaging system components.

The ultrasound-imaging system 1110 further includes ports 1152 for connection with additional components such as optional components 1154 including a printer, storage media, keyboard, or the like, as well as an optional speaker 1155. The ports in some embodiments are universal serial bus (“USB”) ports, though other port types or a combination of port types can be used for this and the other interfaces connections described herein. A power connection 1156 is included with the console 1120 to enable operable connection to an external power supply 1158. An internal battery 1160 can also be employed, either with or exclusive of the external power supply 1158. Power management circuitry 1159 is included with the digital controller/analog interface 1124 of the console to regulate power use and distribution.

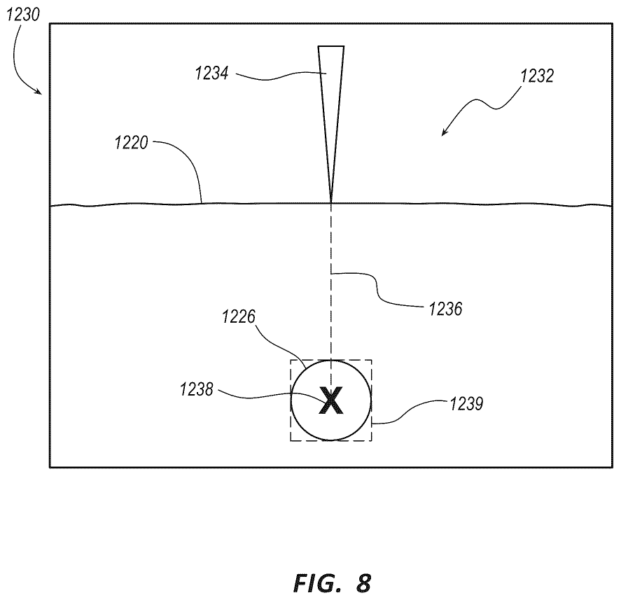

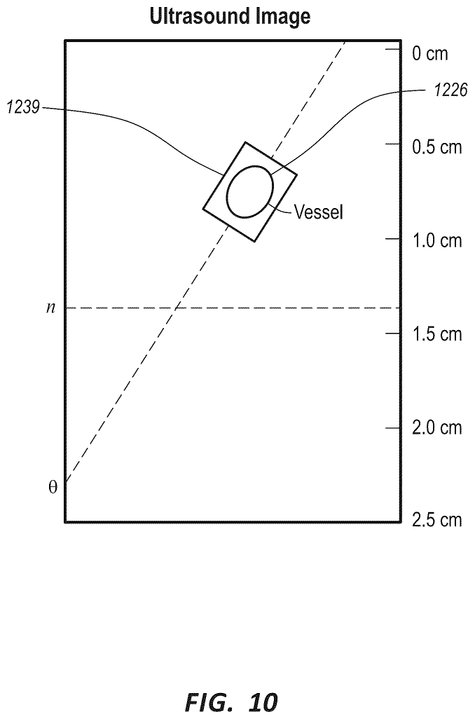

The display 1130 in some embodiments is integrated into the console 1120 and is used to display information to the clinician during the placement procedure, such as an ultrasound image of the targeted internal body portion attained by the probe 1140. For example, FIGS. 8-10 illustrate simplified screen shots from the display 1130 showing a position and orientation of a needle in accordance with ultrasound imaging and sensing a needle as set forth herein. FIG. 10, specifically, illustrates an ultrasound image including the position and orientation of a needle. In some embodiments, the display may be separate from the console 1120. In some embodiments, a console button interface 1132 and control buttons 1184 (FIG. 1) included on the probe 1140 can be used to immediately call up a desired mode to the display 1130 by the clinician to assist in the placement procedure. In some embodiments, the display 1130 is an LCD device.

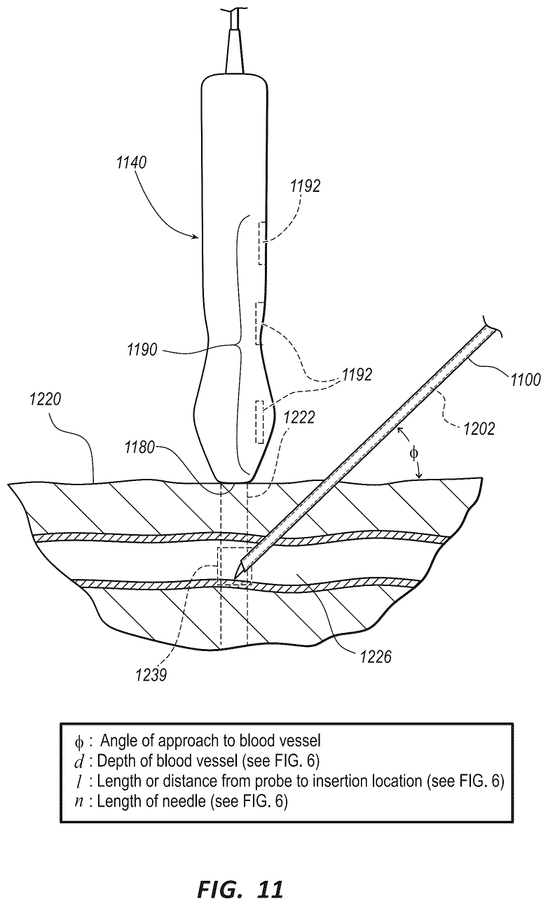

FIG. 1 further depicts a needle 1200 used to gain initial access to the patient vasculature through a skin insertion site. As will be described in further detail below, the needle 1200 is configured to cooperate with the ultrasound-imaging system 1110 in enabling the ultrasound-imaging system 1110 to detect the position, orientation, and advancement of the needle during an ultrasound-based placement procedure. While not shown in FIG. 1, a catheter 1100 such as a short-length catheter can be configured to cooperate with both the needle 1200 and the ultrasound-imaging system 1110 as set forth below. (See, for example, FIGS. 11, 13, and 14.)

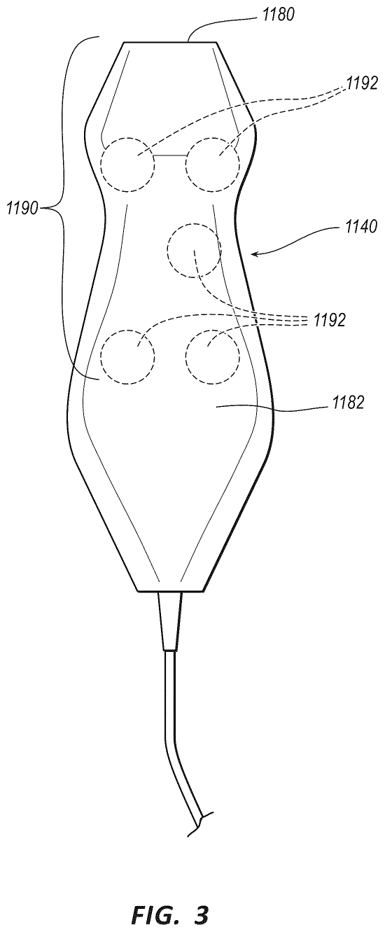

FIG. 3 depicts features of the probe 1140 according to some embodiments. The probe 1140 is employed in connection with ultrasound-based visualization of a blood vessel, such as a vein, in preparation for insertion of the needle 1200, the catheter 1100, or a combination of both the needle 1200 and the catheter 1100 into the vasculature. Such visualization gives real time ultrasound guidance and assists in reducing complications typically associated with such introduction, including inadvertent arterial puncture, hematoma, pneumothorax, etc. The handheld probe 1140 includes a head 1180 that houses a piezoelectric array for producing ultrasonic pulses and for receiving echoes thereof after reflection by the patient's body when the head is placed against the patient's skin proximate the prospective insertion site. The probe 1140 further includes a plurality of control buttons 1184 (FIG. 1) for controlling the ultrasound-imaging system 1110, thus eliminating the need for the clinician to reach out of the sterile field, which is established about the patient insertion site prior to establishment of the insertion site, to control the ultrasound-imaging system 1110.

As such, in some embodiments a clinician employs the ultrasound imaging portion of the ultrasound-imaging system 1110 to determine a suitable insertion site and establish vascular access, such as with the needle 1200, simultaneously with or prior to introduction of a catheter (e.g., the catheter 1100) for ultimate advancement of the catheter 1100 through the vasculature toward an intended destination.

FIG. 2 shows that the probe 1140 further includes a button and memory controller 1142 for governing button and probe operation. The button and memory controller 1142 can include non-volatile memory, such as EEPROM, in some embodiments. The button and memory controller 1142 is in operable communication with a probe interface 1144 of the console 1120, which includes a piezo input/output component 1144A for interfacing with the probe piezoelectric array and a button and memory input/output component 1144B for interfacing with the button and memory controller 1142.

As seen in FIG. 3, the probe 1140 includes a sensor array 1190 for detecting the position, orientation, and movement of the needle 1200 or another medical device during ultrasound imaging procedures, such as those described above. As will be described in further detail below, the sensor array includes a plurality of magnetic sensors 1192 embedded within the housing of the probe 1140. The sensors 1192 are configured to detect a magnetic field associated with the needle 1200 or another medical device and enable the ultrasound-imaging system 1110 to track the needle 1200 or the other medical device. Though configured here as magnetic sensors, it is appreciated that the sensors 1192 can be sensors of other types and configurations, as will be described. Also, though they are shown in FIG. 3 as included with the probe 1140, the sensors 1192 of the sensor array 1190 can be included in a component separate from the probe 1140, such as a separate handheld device. In some embodiments, the sensors 1192 are disposed in a planar configuration below a top face 1182 of the probe 1140, though it is appreciated that the sensors can be arranged in other configurations, such as in an arched or semi-circular arrangement.

In some embodiments, each of the sensors 1192 includes three orthogonal sensor coils for enabling detection of a magnetic field in three spatial dimensions. Such three dimensional (“3-D”) magnetic sensors can be purchased, for example, from Honeywell Sensing and Control of Morristown, N.J. Further, the sensors 1192 of some embodiments are configured as Hall-effect sensors, though other types of magnetic sensors could be employed. Further, instead of 3-D sensors, a plurality of one-dimensional magnetic sensors can be included and arranged as desired to achieve 1-, 2-, or 3-D detection capability.

In some embodiments, five sensors 1192 are included in the sensor array 1190 so as to enable detection of the needle 1200 in not only the three spatial dimensions (i.e., X, Y, Z coordinate space), but also the pitch and yaw orientation of the needle 1200 or another medical device itself. Note that in some embodiments, orthogonal sensing components of two or more of the sensors 1192 enable the pitch and yaw attitude of a magnetic element 1210 of the needle 1200, and thus the needle 1200, itself, to be determined. The orthogonal sensing components of two or more of the sensors 1192 likewise enable the pitch and yaw attitude of a magnetic element of another medical device to be likewise determined.

In some embodiments, fewer or more sensors can be employed in the sensor array 1190. More generally, it is appreciated that the number, size, type, and placement of the sensors 1192 of the sensor array 1900 can vary from what is explicitly shown here.

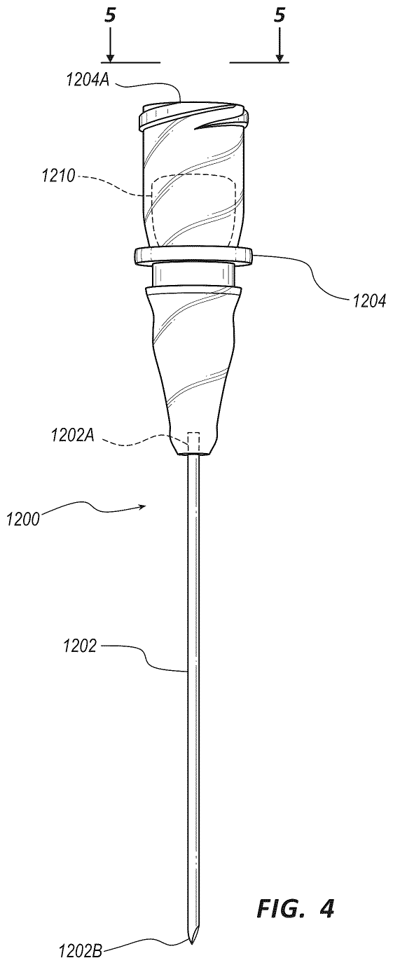

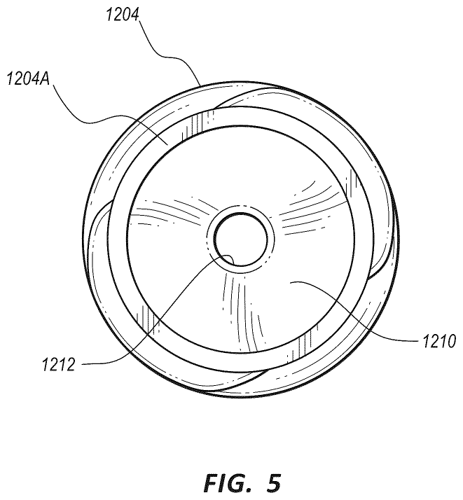

FIGS. 4 and 5 show details of one example of the needle 1200 that can be used in connection with the ultrasound-imaging system 1110 in accessing a targeted internal body portion of the patient, as shown in FIG. 1, according to some embodiments. In particular, the needle 1200 includes a hollow cannula 1202, which defines a proximal end 1202A and a distal end 1202B. A hub 1204 is attached to the proximal end 1202A of the cannula 1202 and includes an open end 1204A that is configured as a connector for connecting with various devices in some embodiments. Indeed, the open end 1204A of the hub 1204 is in fluid communication with the hollow cannula 1202 such that a guide wire, stylet, or other component may be passed through the hub into the cannula 1202.

As shown in FIGS. 4 and 5, a magnetic element 1210 is included with the hub 1204. As best seen in FIG. 5, the magnetic element 1210 in some embodiments is a permanent magnet, including a ferromagnetic substance for instance, and is ring-shaped so as to define hole 1212 that is aligned with the hollow cannula 1202. So configured, the magnetic element 1210 produces a magnetic field that is detectable by the sensor array 1190 of the ultrasound probe 1140 so as to enable the location, orientation, and movement of the needle 1200 to be tracked by the ultrasound-imaging system 1110, as described further below.

In some embodiments, it is appreciated that many other types, numbers, and sizes of magnetic elements can be employed with the needle 1200 or other medical devices (e.g. catheters) to enable tracking thereof by the ultrasound-imaging system 1110.

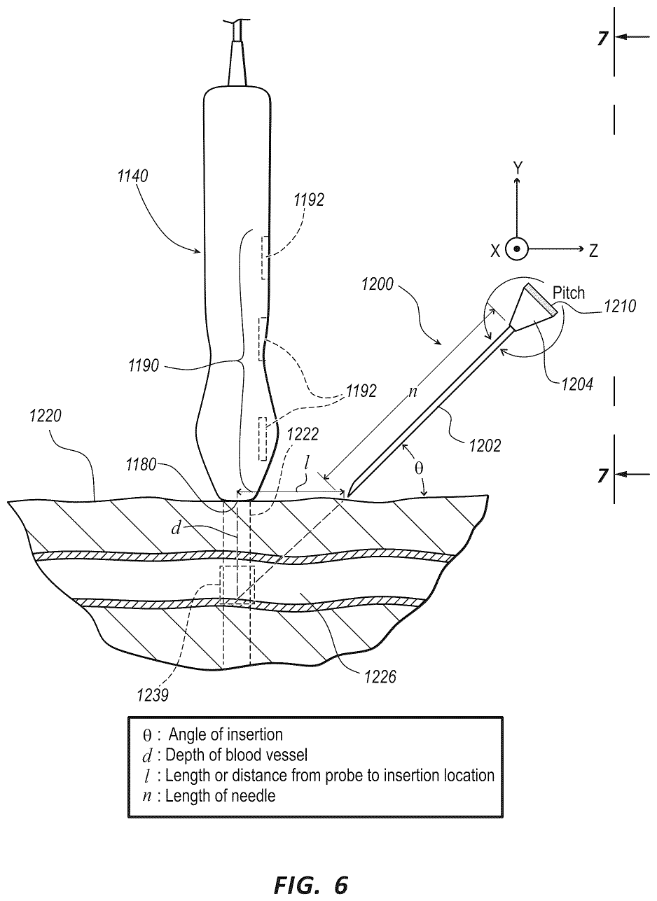

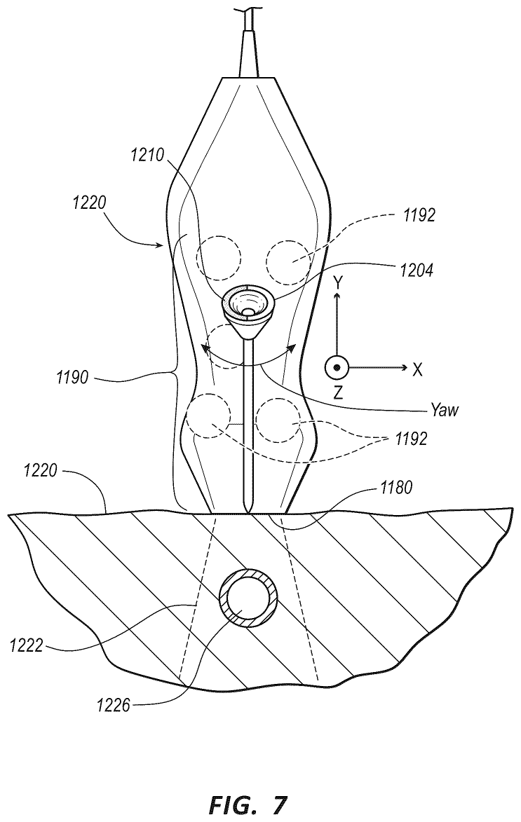

Reference is now made to FIG. 6 and FIG. 7, which show the ultrasound probe 1140 of the ultrasound-imaging system 1110 and the needle 1200 in position and ready for insertion thereof through a skin surface 1220 of a patient to access a targeted internal body portion (e.g., a portion of a blood vessel 1226). In particular, the probe 1140 is shown with its head 1180 placed against the skin surface 1220 and producing an ultrasound beam 1222 so as to ultrasonically image a portion of the portion of the blood vessel 1226 beneath the skin surface 1220 of the patient. The ultrasonic image of the blood vessel 1226 can be depicted on the display 1130 of the ultrasound-imaging system 1110 (FIG. 1).

As mentioned above, the ultrasound-imaging system 1110 in some embodiments is configured to detect the position, orientation, and movement of the needle 1200 described above. In particular, the sensor array 1190 of the probe 1140 is configured to detect a magnetic field of the magnetic element 1210 included with the needle 1200. Each of the sensors 1192 of the sensor array 1190 is configured to spatially detect the magnetic element 1210 in three-dimensional space. Thus, during operation of the ultrasound-imaging system 1110, magnetic field strength data of the needle's magnetic element 1210 sensed by each of the sensors 1192 is forwarded to a processor, such as the processor 1122 of the console 1120 (FIG. 2), which computes in real-time the position, orientation, or both the position and orientation of the magnetic element 1210.

Specifically, as shown in FIG. 6 and FIG. 7, the position of the magnetic element 1210 in X, Y, and Z coordinate space with respect to the sensor array 1190 can be determined by the ultrasound-imaging system 1110 using the magnetic field strength data sensed by the sensors 1192. Moreover, FIG. 6 shows that the pitch of the magnetic element 1210 can also be determined, while FIG. 7 shows that the yaw of the magnetic element 1210 can be determined. Suitable logic (e.g., the logic components 1121) cooperating with the processor 1122 or other suitable components of the ultrasound-imaging system 1110 can provide the calculations necessary for such position, orientation, or both position and orientation. In some embodiments, the magnetic element 1210 can be tracked using the teachings of one or more of the following U.S. patents, each of which is incorporated by reference in its entirety into this application: U.S. Pat. Nos. 5,775,322; 5,879,297; 6,129,668; 6,216,028; and 6,263,230.

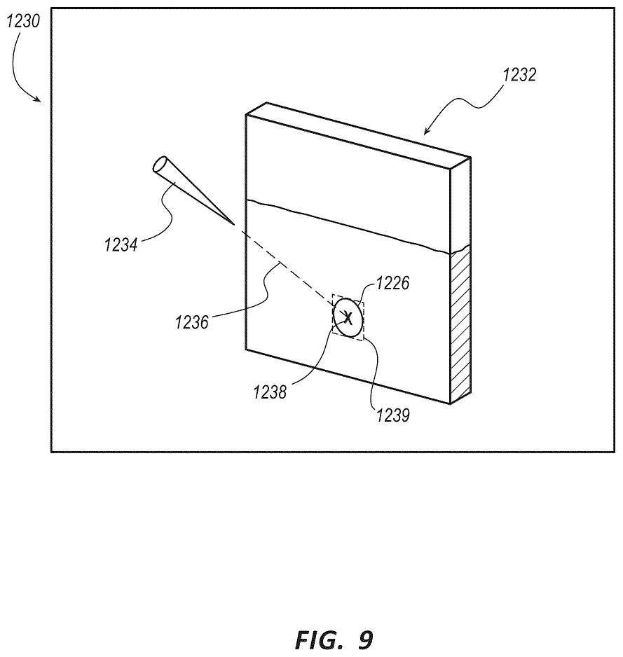

The above position and orientation information determined by the ultrasound-imaging system 1110, together with the length of the cannula 1202 and position of the magnetic element 1210 with respect to the distal needle tip as known by or input into the ultrasound-imaging system 1110, enable the ultrasound-imaging system 1110 to accurately determine the location and orientation of the entire length of the needle 1200 with respect to the sensor array 1190. Optionally, the distance between the magnetic element 1210 and the distal needle tip is known by or input into the ultrasound-imaging system 1110. This in turn enables the ultrasound-imaging system 1110 to superimpose an image of the needle 1200 on to an image produced by the ultrasound beam 1222 of the probe 1140. FIGS. 8 and 9 show examples of such a superimposition of the needle 1200 onto an ultrasound image. FIG. 10 shows an alternative example in which a distinct (e.g., dotted, colored, etc.) and dynamic line over the ultrasound image changes in accordance with the angle of insertion θ, depth of the blood vessel d, an insertion location above the blood vessel defined by the distance or length (from the probe 1140 to the insertion location, and known length η of the needle 1200. (See, also, FIG. 6 for the angle of insertion θ, the depth of the blood vessel d, the distance or length (from the probe 1140 to the insertion location, and known length η of the needle 1200.)

Specifically, FIGS. 8 and 9 each show a screenshot 1230 that can be depicted on the display 1130 (FIG. 1), for instance. In FIG. 8, an ultrasound image 1232 is shown, including depiction of the patient skin surface 1220, and the subcutaneous blood vessel 1226. The ultrasound image 1232 corresponds to an image acquired by the ultrasound beam 1222 shown in FIG. 6 and FIG. 7, for instance. The screenshot 1230 further shows a needle image 1234 representing the position and orientation of the actual needle 1200 as determined by the ultrasound-imaging system 1110 as described above. Because the ultrasound-imaging system 1110 is able to determine the location and orientation of the needle 1200 with respect to the sensor array 1190, the ultrasound-imaging system 1110 is able to accurately determine the position and orientation of the needle 1200 with respect to the ultrasound image 1232 and superimpose it thereon for depiction as the needle image 1234 on the display 1130. Coordination of the positioning of the needle image 1234 on the ultrasound image 1232 is performed by suitable logic (e.g., the logic components 1121) cooperating with the processor 1122 or other suitable component of the ultrasound-imaging system 1110.

The sensors 1192 are configured to continuously detect the magnetic field of the magnetic element 1210 of the needle 1200 during operation of the ultrasound-imaging system 1110. This enables the ultrasound-imaging system 1110 to continuously update the position and orientation of the needle image 1234 for depiction on the display 1130. Thus, advancement or other movement of the needle 1200 is depicted in real-time by the needle image 1234 on the display 1130. Note that the ultrasound-imaging system 1110 is capable of continuously updating both the ultrasound image 1232 and the needle image 1234 on the display 1130 as movements of the probe 1140 and the needle 1200 occur during a placement procedure or other activity.

FIG. 8 further shows that in some embodiments the ultrasound-imaging system 1110 can depict a projected path 1236 based on the current position and orientation of the needle 1200 as depicted by the needle image 1234. The projected path 1236 assists a clinician in determining whether the current orientation of the needle 1200, as depicted by the needle image 1234 on the display 1130, will result in arriving at the targeted internal body portion such as the blood vessel 1226 shown here. Again, as the orientation or position of the needle image 1234 changes, the projected path 1236 is correspondingly modified by the ultrasound-imaging system 1110. A target 1238, indicating the point where the projected path 1236 crosses the plane of the ultrasound image 1232, can also be depicted on the display 1130 by the ultrasound-imaging system 1110. As shown in FIG. 8, the target 1238 is located within the blood vessel 1226 depicted in the ultrasound image 1232. Note that the position of the target 1238 on the display 1130 can also be modified as the needle 1200 or the ultrasound image 1232 are adjusted. The screenshot 1230 also includes an area of probability 1239, here depicted as a box, which indicates any possible margin of error of the ultrasound-imaging system 1110 due to needle length, needle rigidity and flex, field strength of the magnetic element, magnetic interference, possible discrepancy in alignment of the magnetic axis of the magnetic element with the longitudinal axis of the needle, orientation of the sensor array with respect to the ultrasound imaging plane, etc. For correspondence, the area of probability 1239 is also depicted in FIGS. 6, 11, 13, and 14.

FIG. 9 shows that, in some embodiments, the screenshot 1230 can be configured such that the ultrasound image 1232 and the needle image 1234 are oriented so as to be displayed in a three-dimensional aspect. This enables the angle and orientation of the needle 1200, as depicted by the needle image 1234, to be ascertained and compared with the intended target imaged by the ultrasound image 1232. It should be noted that the screenshots 1230 are merely examples of possible depictions produced by the ultrasound-imaging system 1110 for display; indeed, other visual depictions can be used. Note further that the particular area of the body being imaged is merely an example; the ultrasound-imaging system 1110 can be used to ultrasonically image a variety of body portions, and should not be limited to what is explicitly depicted in the accompanying figures. Further, the ultrasound-imaging system 1110 as depicted and described herein can be included as a component of a larger system, if desired, or can be configured as a stand-alone device. Also, it is appreciated that, in addition to the visual display 1130, aural information, such as beeps, tones, etc., can also be employed by the ultrasound-imaging system 1110 to assist the clinician during positioning and insertion of the needle 1200 into the patient.

As mentioned above, in some embodiments it is necessary for the ultrasound-imaging system 1110 to know the total length of the needle 1200 and the location of the magnetic element 1210 thereon in order to enable an accurate depiction of the needle image 1234 and other features of the screenshots 1230 of FIGS. 8 and 9 to be made. The ultrasound-imaging system 1110 can be informed of these or other pertinent parameters in various ways, including scanning by the ultrasound-imaging system 1110 of a barcode included on or with the needle 1200, the inclusion of a radiofrequency identification (“RFID”) chip with the needle 1200 for scanning by the ultrasound-imaging system 1110, color coding of the needle 1200, manual entry of the parameters by the clinician into the ultrasound-imaging system 1110, etc. Likewise, the ultrasound-imaging system 1110 can be informed of pertinent parameters for other medical devices (e.g., the catheter 1100) in the foregoing ways (e.g., scanning by the ultrasound-imaging system 1110 of a barcode included on or with the other medical device, the inclusion of an RFID chip with the other medical device for scanning by the ultrasound-imaging system 1110, color coding of the other medical device, manual entry of the parameters by the clinician into the ultrasound-imaging system 1110, etc.) The probe 1140 or other component of the ultrasound-imaging system 1110 can include an RFID reader to read information included on the RFID chip of the needle 1200 or another medical device, such as the type or length of the needle 1200, the catheter 1100, etc. These and other means for inputting the needle and other parameters into the ultrasound-imaging system 1110 or detecting the parameters are therefore contemplated.

In some embodiments, a length of the needle 1200 (or other aspect of a medical device such as the catheter 1100) can be measured by the probe 1140 and ultrasound-imaging system 1110 using a characteristic of the magnetic field of the needle 1200, such as the magnetic poles, magnetic field shape, magnetic field strength, etc. For instance, in some embodiments the magnetic element 1210 of the needle 1200 can be positioned at a predetermined distance from the probe 1140 or at a predetermined location with respect to the probe 1140. With the magnetic element 1210 so positioned, the sensor array 1190 of the probe 1140 detects and measures the field strength of the magnetic element 1210, the cannula 1202, or a combination thereof. The ultrasound-imaging system 1110 can compare the measured field strength with a stored list of possible field strengths corresponding to different lengths of needles. The ultrasound-imaging system 1110 can match the two strengths and determine the needle length. The needle location and subsequent needle insertion can then proceed as described herein. In some embodiments, instead of holding the magnetic element 1210 stationary at a predetermined location, the magnetic element 1210 can be moved about the probe 1140 such that multiple field strength readings are taken by the probe 1140. Aspects that can be modified so as to impart different field strengths to a set of magnetic element include size, shape, and composition of the magnetic element 1210, etc.

Further details are given here regarding use of the ultrasound-imaging system 1110 in guiding the needle 1210 or other medical device (e.g., the catheter 1100) in connection with ultrasonic imaging of a targeted internal body portion (“target”) of a patient, according to some embodiments. With the magnetic element-equipped needle 1200 positioned a suitable distance (e.g., two or more feet) away from the ultrasound probe 1140 including the sensor array 1190, the probe 1140 is employed to ultrasonically image, for depiction on the display 1130 of the ultrasound-imaging system 1110, the target within the patient that the needle is intended to intersect via percutaneous insertion. A calibration of the ultrasound-imaging system 1110 is then initiated, in which logic (e.g., the logic components 1121) cooperates with the processor 1122 of the console 1120 to determine a baseline for any ambient magnetic fields in the vicinity of where the procedure will be performed. The ultrasound-imaging system 1110 is also informed of the total length of the needle 1200, or position of the magnetic element 1210 with respect to the distal needle tip such as by user input, automatic detection, or in another suitable manner, as has been discussed above.

The needle 1200 is then brought into the range of the sensors 1192 of the sensor array 1190 of the probe 1140. Each of the sensors 1192 detects the magnetic field strength associated with the magnetic element 1210 of the needle 1200, which data is forwarded to the processor 1122. In some embodiments, such data can be stored in the memory 1123 until needed by the processor 1122. As the sensors 1192 detect the magnetic field, suitable logic (e.g., the logic components 1121) cooperates with the processor 1122 to calculate a magnetic field strength of the magnetic element 1210 of the needle 1200 at predicted points in space in relationship to the probe 1140. The processor 1122 then compares the actual magnetic field strength data detected by the sensors 1192 to the calculated field strength values. This process is further described by the U.S. patents identified herein. This process can be iteratively performed until the calculated value for a predicted point matches the measured data. Once this match occurs, the magnetic element 1210 has been positionally located in three-dimensional space. Using the magnetic field strength data as detected by the sensors 1192, the pitch and yaw (i.e., orientation) of the magnetic element 1210 can also be determined. Together with the known length of the needle 1200 and the position of the distal tip of the needle 1200 with respect to the magnetic element 1210, this enables an accurate representation of the position and orientation of the needle 1200 can be made by the ultrasound-imaging system 1110 and depicted as a virtual model, i.e., the needle image 1234, on the display 1130. Note that the predicted and actual detected values must match within a predetermined tolerance or confidence level in some embodiments for the ultrasound-imaging system 1110 to enable needle depiction to occur.

Depiction of the virtual needle image 1234 of the needle 1200 as described above is performed in some embodiments by overlaying the needle image 1234 on the ultrasound image 1232 of the display 1130 (FIGS. 8 and 9). Suitable logic (e.g., the logic components 1121) of the ultrasound-imaging system 1110 as executed by the processor 1122 or other suitable component further enable the projected path 1236, the target 1238, and area of probability 1239 (FIGS. 8 and 9) to be determined and depicted on the display 1130 atop the ultrasound image 1232 of the target. The above prediction, detection, comparison, and depiction process is iteratively performed to continue tracking the movement of the needle 1200 in real-time.

The needle 1200 represents an example of a medical device the ultrasound-imaging system 1110 is configured to locate and guide during ultrasound-based access of a subcutaneous blood vessel of a patient with the needle 1200. It should be understood that other medical devices such as the catheter 1100 can be configured with features like the needle 1200 for location and guiding by the ultrasound-imaging system 1110. Indeed, insofar as features of the needle 1200 are needed by another medical device such as the catheter 1100 for cooperation with the ultrasound-imaging system 1110, those features are included in the other medical device. Thus, this disclosure is extended without burdening the disclosure. However, it should also be understood that interpretation of the disclosure in the foregoing manner does not extend to the claims. For example, a claimed needle shall not read on an existing catheter, a claimed catheter shall not read on an existing needle, and so on.

For modalities other than the foregoing magnetic-based modality, including optical modalities, radiofrequency electromagnetic radiation-based modalities, and radioactive modalities, see U.S. Pat. No. 9,492,097, which is incorporated by reference in its entirety into this application.

### Methods

### Methods for Accessing a Blood Vessel

FIG. 6 illustrates a first view of the ultrasound probe 1140 of the ultrasound-imaging system 1110 being used to guide percutaneous insertion of the needle 1200 into the blood vessel 1226 of a patient in accordance with some embodiments.

Methods for accessing a blood vessel (e.g., the blood vessel 1226) with a medical device (e.g., the needle 1200, the catheter 1100, etc.) include a set of operations performed by executing instructions of a non-transitory computer-readable medium (“CRM”) such as the memory 1123 by one or more processors (e.g., the processor 1122) of the ultrasound-imaging system 1110 that cause the ultrasound-imaging system 1110 to perform the set of operations, which include, in some embodiments, determining a depth of the blood vessel d with vessel depth-determination logic of the logic components 1121 using data from the ultrasound probe 1140 gathered above the blood vessel 1226 as input; calculating whether the medical device is able to access the blood vessel 1226 with medical device-accessibility logic of the logic components 1121 using the depth of the blood vessel d, an effective length η of the medical device, an insertion location above the blood vessel 1226 defined by the distance or length l from the probe 1140 to the insertion location, and an insertion angle θ of the medical device as inputs, wherein the calculating can include triangulation as shown in FIG. 6; and displaying a visual indicator on the display 1130 such as over an ultrasound image, (see, for example, FIGS. 8-10) emitting an audio indicator from the speaker 1155, or both to indicate whether the medical device will be able to access the blood vessel 1226. Each indicator of the visual indicator and the audio indicator is configured to be toggled on or off by a user of the ultrasound-imaging system 1110.

The set of operations can further include determining the distance or length l from the probe 1140 to the insertion location and the insertion angle θ of the medical device from sensor readings from the plurality of magnetic sensors 1192 of the ultrasound probe 1140 configured to detect the medical device such as by an associated magnetic field.

The visual indicator can be a target overlying the ultrasound image on the display 1130, which target can fade away, vanish, switch from one color (e.g., green) to another color (e.g., yellow), or switch from one pattern to another pattern to indicate the medical device is not able to access the blood vessel 1226. Alternatively, the visual indicator can be an elongate graphical element overlying the ultrasound image that represents the effective length of the medical device. (See, for example, FIGS. 8-10.)

Methods for Ensuring Placement of a Sufficient Length is within a Blood Vessel

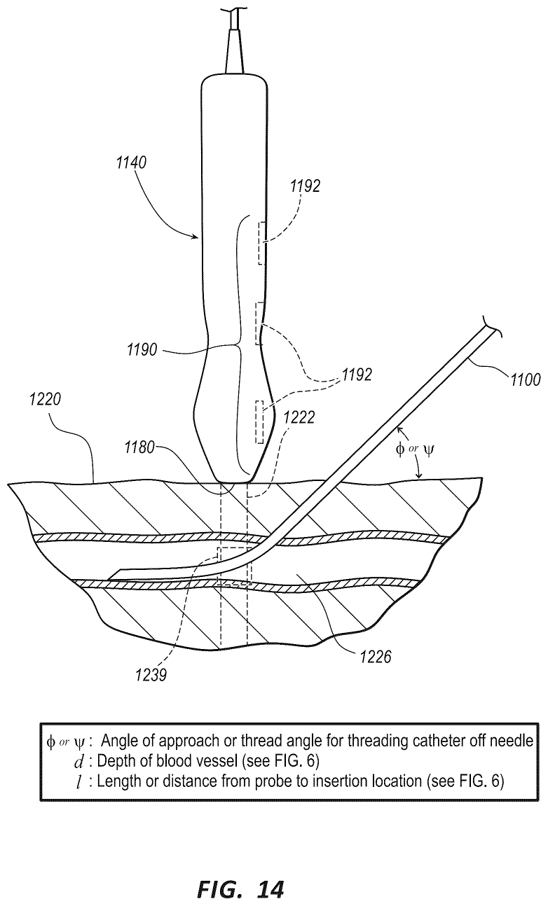

FIG. 14 illustrates a view of the ultrasound probe 1140 of the ultrasound-imaging system 1110 being used to calculate an amount of the catheter 1100 in the blood vessel 1226 of a patient in accordance with some embodiments.

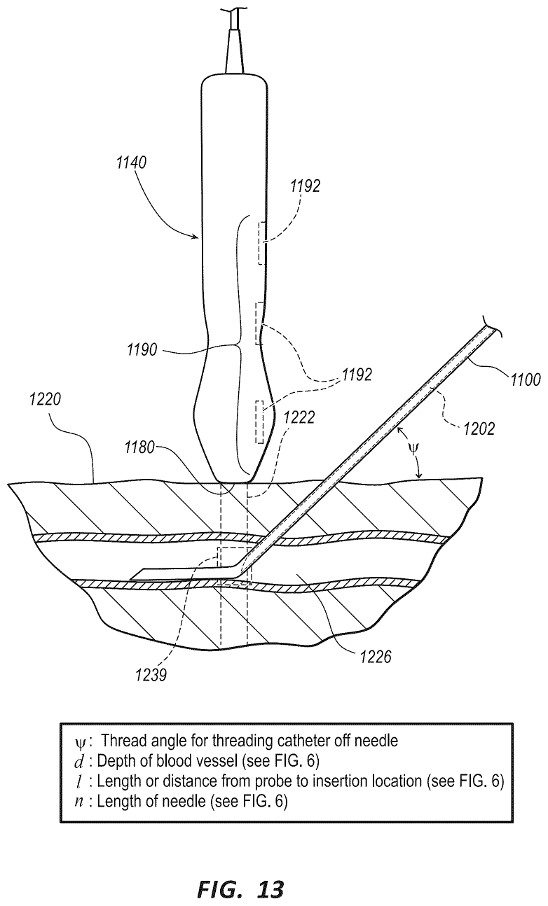

Methods for ensuring a final placement of a sufficient length of a medical device (e.g., the needle 1200, a short-length catheter such as peripheral intravenous line having a magnetized distal end portion, for example, the catheter 1100, etc.) is within a blood vessel (e.g., the blood vessel 1226) include a set of operations performed by executing instructions of a non-transitory CRM such as the memory 1123 by one or more processors (e.g., the processor 1122) of the ultrasound-imaging system 1110 that cause the ultrasound-imaging system 1110 to perform the set of operations, which include, in some embodiments, determining the depth of the blood vessel d with vessel depth-determination logic of the logic components 1121 using data from the ultrasound probe 1140 gathered above the blood vessel 1226 as input; calculating whether a minimum length of the medical device is or will be placed within the blood vessel 1226 with medical device-placement logic of the logic components 1121 from the depth of the blood vessel d, the effective length η of the medical device, the insertion location above the blood vessel 1226 defined by the distance or length (from the probe 1140 to the insertion location, and an angle of approach φ of the medical device (see FIG. 11) or thread angle ψ (see FIG. 13) for threading the catheter 1100 off the needle 1200 as inputs, wherein the calculating can include triangulation akin to that shown in FIG. 6 to determine the length of the medical device disposed subcutaneously (e.g., along the hypotenuse of the triangle) and subtraction along with an exposed length of the medical device from the effective length η of the medical device to find the length of the medical device within the blood vessel 1226; displaying a visual indicator on the display 1130 such as over an ultrasound image, emitting an audio indicator from the speaker 1155, or both to indicate whether a potential placement of the medical will result in the final placement of a sufficient length of the medical device within the blood vessel 1226.

The set of operations can further include determining the distance or length (from the probe 1140 to the insertion location and the angle of approach φ of the medical device or thread angle ψ for threading the catheter 1100 off the needle 1200 from sensor readings from the plurality of magnetic sensors 1192 of the ultrasound probe 1140 configured to detect the medical device such as by an associated magnetic field.

As an alternative to triangulation akin to that shown in FIG. 6 to determine the length of the medical device disposed subcutaneously followed by subtraction along with the exposed length of the medical device from the effective length η of the medical device to find the length of the medical device within the blood vessel 1226, triangulation can be used to directly determine the length of the medical device within the blood vessel 1226 from inputs including the location of the probe 1140 on the skin surface 1220, the depth of the blood vessel d, and a magnetic signal from the tip of the catheter 1100 when the tip of the catheter 1100 includes a magnetic element. In any case, the ultrasound-imaging system 1110 can utilize length-determination logic of the logic components 1121 to indicate whether the length of the medical device within the blood vessel 1226 is sufficient for the procedure or the medical device.

The visual indicator can be a target overlying the ultrasound image on the display 1130, which target can fade away, vanish, switch from one pattern to another pattern, or switch from one color (e.g., green) to another color (e.g., yellow) to indicate the potential placement of the medical device will not result in the final placement of a sufficient length of the medical device within the blood vessel 1226. The minimum length of the medical device can be user defined (e.g. 1 inch of the medical device at final placement), or the minimum length can be set in accordance with a known, or suggested, minimum length provided by a manufacturer of the medical device.

When the medical device is a short-length catheter, such as the catheter 1100, having a magnetized distal end portion, the set of operations can further include estimating a distance the distal-end portion, or a tip thereof, of the catheter 1100 is from a tip of a needle in the blood vessel 1226 with tip-estimation logic of the logic components 1121 as the catheter 1100 is advanced over the needle. (See FIG. 13.) Alternatively, the catheter 1100 can have a magnet in a hub of the catheter 1100, and the set of operations can further include estimating a distance the magnetized hub of the catheter 1100 is from a tip of a needle in the blood vessel 1226 with tip-estimation logic of the logic components 1121 as the catheter 1100 is advanced over the needle. The estimation of the distance can be displayed on the display 1130 over an ultrasound image, which allows a user such as a clinician to, for example, lower the angle of approach φ of the medical device and track subsequent advancement of the catheter 1100 (e.g., 2 mm in the blood vessel 1226 or beyond the needle).

Again, the visual indicator can be a target overlying the ultrasound image on the display 1130, which target can include a catheter icon to dynamically show the catheter 1100 in the blood vessel 1226. The catheter icon can be configured to vanish when the user starts advancing the catheter 1100 so as to not obscure the target.

### Methods for Recommending a Proper Approach Angle

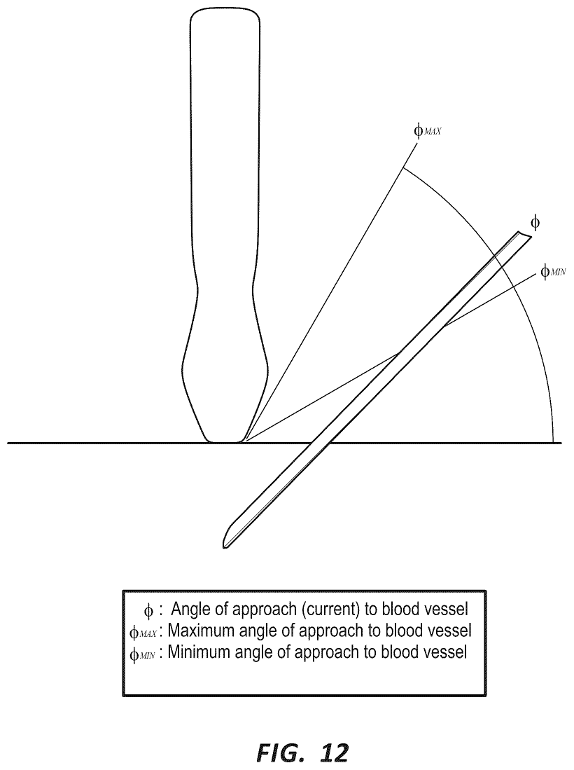

FIG. 11 illustrates a view of the ultrasound probe of the ultrasound-imaging system 1110 being used to guide insertion of a combination of the catheter 1100 and the needle 1200 into the blood vessel 1226 of a patient in accordance with some embodiments. FIG. 12 illustrates a simplified version of a screenshot from the display 1130 of the ultrasound-imaging system 1110 graphically showing a guide for the angle of approach φ for insertion of the combination of the catheter 1100 and the needle 1200 into the blood vessel 1226 of the patient in accordance with some embodiments. It should be understood the angle of insertion θ and the angle of approach φ are different in that the angle of insertion θ is directed to a percutaneous insertion, whereas the angle of approach φ is directed to approaching, for example, the blood vessel 1226 after the percutaneous insertion. Often, the angle of approach φ is less than the angle of insertion θ, thereby increasing the probability the medical device enters a lumen of the blood vessel 1226 and mitigating the risk the medical device passes entirely thorough the blood vessel 1226.

Methods for recommending a proper approach angle φ for approaching a blood vessel (e.g., the blood vessel 1226) with a medical device (e.g., a needle, a short-length catheter such as peripheral intravenous line having a magnetized distal end portion, etc.) include a set of operations performed by executing instructions of a non-transitory CRM such as the memory 1123 by one or more processors (e.g., the processor 1122) of the ultrasound-imaging system 1110 that cause the ultrasound-imaging system 1110 to perform the set of operations, which include, in some embodiments, determining a presence of the medical device from sensor readings from the plurality of magnetic sensors 1192 of the ultrasound probe 1140 configured to detect the medical device such as by an associated magnetic field; and displaying a visual indicator on the display 1130 (see, for example, FIG. 9) such as over an ultrasound image to indicate the proper approach angle φ for approaching the blood vessel 1226 with the medical device.

The set of operations can further include determining a trajectory of the medical device with trajectory-determination logic of the logic components1121 using the sensor readings as input. For example, the trajectory-determination logic can utilize triangulation akin to that shown in FIG. 6 for determining the angle of approach φ instead of the angle of insertion θ. The trajectory of the medical device can be displayed on the display 1130 over an ultrasound image. In addition, the visual indicator can be incorporated into the trajectory of the medical device on the display 1130 over the ultrasound image.

The set of operations can further include issuing a visible warning on the display 1130 over the ultrasound image or an audible warning from the speaker 1155 if the trajectory is determined to include a large approach angle φ (e.g., 90°) to the blood vessel 1226. The visible or audible warning can also be issued if the trajectory is determined to pass through an artery.

The visual indicator over the ultrasound image on the display 1130 can switch from one pattern to another pattern, one color (e.g., green for OK) to another color (e.g., yellow for too steep), or from a dashed line to a solid line to indicate the trajectory of the medical device follows the proper approach angle φ for approaching the blood vessel 1226 with the medical device. (See, for example, FIG. 9.) Numerical readings for the insertion angle θ can also be displayed over the ultrasound image on the display 1130. As shown in FIG. 12, the visual indicator can be like the needle image 1234 of FIGS. 8 and 9 but superimposed over a minimum recommended angle of approach φₘᵢₙ and a maximum recommended angle of approach φₘₐₓ.

The proper approach angle φ can be set in accordance with a recommendation by a manufacturer of the medical device, an established medical procedure for the medical device, or a user's preference for using the medical device. Such approach angles φ can be based on design, laboratory studies, or clinical evaluations as certain approach angles φ to a blood vessel can be advantageous for access to reduce difficulties (e.g., ease of threading) or complications (e.g., kinking, backwalling, etc.).

### Methods for Recommending a Proper Thread Angle

FIG. 13 illustrates a view of the ultrasound probe 1140 of the ultrasound-imaging system 1110 being used to guide threading of the catheter 1100 off the needle 1200 into the blood vessel 1226 of a patient in accordance with some embodiments.

Methods for recommending a proper thread angle ψ for threading the catheter 1100 off the needle 1200 into a blood vessel (e.g., the blood vessel 1226) include a set of operations performed by executing instructions of a non-transitory CRM such as the memory 1123 by one or more processors (e.g., the processor 1122) of the ultrasound-imaging system 1110 that cause the ultrasound-imaging system 1110 to perform the set of operations, which include, in some embodiments, determining a presence of the medical device (i.e., the combination of the catheter 1100 and the needle 1200) from sensor readings from the plurality of magnetic sensors 1192 of the ultrasound probe 1140 configured to detect the medical device such as by an associated magnetic field; and displaying a visual indicator on the display 1130 (see, for example, FIG. 9) such as over an ultrasound image to indicate the proper thread angle ψ for threading the catheter 1100 off the needle 1200 into the blood vessel 1226. Between determining the presence of the medical device from the sensor readings and displaying the visual indicator on the display 1130, the ultrasound-imaging system 1110 can be configured to determine, as part of the set of operations, a depth of the blood vessel d with the vessel depth-determination logic of the logic components 1121 using data from the ultrasound probe 1140 gathered above the blood vessel 1226 as input, as well as calculate the thread angle ψ by triangulation as set forth herein using inputs including at least the depth of the blood vessel d, the effective length η of the medical device (e.g., the catheter 1100, the needle 1200, or both), and an insertion location above the blood vessel 1226 defined by the distance or length (from the probe 1140 to the insertion location as shown in FIG. 6.

The set of operations can further include issuing a visible warning over the ultrasound image on the display 1130 or an audible warning from the speaker 1155 if the thread angle ψ is determined to be too large or small.

The visual indicator can switch from one pattern to another pattern, one color (e.g., green for OK) to another color (e.g., yellow for too steep or shallow), or from a dashed line to a solid line to indicate the thread angle ψ is proper for threading the catheter 1100 off the needle 1200 into the blood vessel 1226. (See, for example, FIG. 9.) Numerical readings for the thread angle ψ can also be displayed over the ultrasound image on the display 1130. As shown in FIG. 12, the visual indicator can be like the needle image 1234 of FIGS. 8 and 9 but superimposed over a minimum recommended thread angle ψₘᵢₙ and a maximum recommended thread angle ψₘₐₓ.

The proper thread angle ψ can be set in accordance with a recommendation by a manufacturer of the medical device, an established medical procedure for the medical device, or a user's preference for using the medical device. Such thread angles ψ can be based on design, laboratory studies, or clinical evaluations as certain thread angles ψ to a blood vessel can be advantageous for access to reduce difficulties (e.g., case of threading) or complications (e.g., kinking, backwalling, etc.).

### Methods for Recommending a Proper Insertion Angle

FIG. 6 illustrates a first view of the ultrasound probe 1140 of the ultrasound-imaging system 1110 being used to guide percutaneous insertion of the needle 1200 into the blood vessel 1226 of a patient in accordance with some embodiments.

Methods for recommending a proper insertion angle θ for inserting a medical device (e.g., a needle, a short-length catheter such as peripheral intravenous line having a magnetized distal end portion, etc.) into a blood vessel (e.g., the blood vessel 1226) include a set of operations performed by executing instructions of a non-transitory CRM such as the memory 1123 by one or more processors (e.g., the processor 1122) of the ultrasound-imaging system 1110 that cause the ultrasound-imaging system 1110 to perform the set of operations, which include, in some embodiments, determining a presence of the medical device from sensor readings from the plurality of magnetic sensors 1192 of the ultrasound probe 1140 configured to detect the medical device such as by an associated magnetic field; and displaying a visual indicator on the display 1130 (see, for example, FIG. 9) such as over an ultrasound image to indicate the proper insertion angle θ for inserting the medical device into the skin surface 1220 and subsequently into the blood vessel 1226. Between determining the presence of the medical device from the sensor readings and displaying the visual indicator on the display 1130, the ultrasound-imaging system 1110 can be configured to determine, as part of the set of operations, a depth of the blood vessel d with the vessel depth-determination logic of the logic components 1121 using data from the ultrasound probe 1140 gathered above the blood vessel 1226 as input, as well as calculate the angle of insertion θ by triangulation as set forth herein using inputs including at least the depth of the blood vessel d, the effective length η of the medical device, and an insertion location above the blood vessel 1226 defined by the distance or length l from the probe 1140 to the insertion location as shown in FIG. 6.

The visual indicator can appear over the ultrasound image on the display 1130 at a time the medical device reaches the blood vessel 1226 but before insertion of the medical device in the blood vessel 1226. (See, for example, FIG. 9.) Alternatively, the visual indicator can be continuously shown over the ultrasound image on the display 1130 with two or more differently colored or patterned zones. One zone of the two or more zones can be enhanced to indicate whether an approach of the medical device is in accordance with the proper insertion angle θ for inserting the medical device in the blood vessel 1226. Numerical readings for the insertion angle θ can also be displayed over the ultrasound image on the display 1130. While FIG. 12 is directed to the angle of approach φ, it could likewise depict the angle of insertion θ. As such, the visual indicator can be like the needle image 1234 of FIGS. 8 and 9 but superimposed over a minimum recommended angle of insertion φₘᵢₙ and a maximum recommended angle of insertion θₘₐₓ.

The proper insertion angle θ can be set in accordance with a recommendation by a manufacturer of the medical device, an established medical procedure for the medical device, or a user's preference for using the medical device. Such insertion angles θ can be based on design, laboratory studies, or clinical evaluations as certain insertion angles θ can be advantageous to reduce difficulties (e.g., ease of threading) or complications (e.g., kinking, backwalling, etc.). Indication of the proper insertion angle θ is advantageous because some medical devices benefit from lowering the insertion angle θ from that of the angle of approach φ.

### Methods for Optimizing an Ultrasound Image

Methods for optimizing an ultrasound image about a blood vessel (e.g., the blood vessel 1226) or a targeted location of the blood vessel 1226 include a set of operations performed by executing instructions of a non-transitory CRM such as the memory 1123 by one or more processors (e.g., the processor 1122) of the ultrasound-imaging system 1110 that cause the ultrasound-imaging system 1110 to perform the set of operations, which include, in some embodiments, detecting the blood vessel 1226 using ultrasound signals echoed off the blood vessel and received by an ultrasound probe; and adjusting one or more parameters of the ultrasound probe 1140 selected from a focus of the ultrasound probe 1140, an operating frequency of the ultrasound probe 1140, and an acoustic power output of the ultrasound probe 1140 above the blood vessel 1226 or the targeted location of the blood vessel 1226, thereby optimizing the ultrasound image about the blood vessel 1226 or the targeted location of the blood vessel 1226. The ultrasound-imaging system 1110 is configured such that the foregoing adjustments are made with minimal noticeable impact to the user such as without significant screen refreshes or other pauses.

The set of operations can further include determining the targeted location from a hysteretic analysis of locations of the ultrasound probe 1140 above the blood vessel 1226. The set of operations can further include determining with blood vessel-occupation logic of the logic components 1121 a percentage of the blood vessel 1226 to be occupied by a medical device (e.g., a needle, a short-length catheter such as peripheral intravenous line having a magnetized distal end portion, etc.) upon insertion of a sufficient length of the medical device in the blood vessel 1226. Determining the percentage of the blood vessel 1226 to be occupied by the medical device can include summation of a number of cross-sectional areas of the blood vessel 1226 for a luminal volume of the blood vessel 1226 for comparison with a known volume of a minimum length of the medical device for placement in the blood vessel 1226.

A pre-assessment magnetic stylet similar to that of U.S. Pat. No. 9,492,097, as well as the medical device (e.g., a needle, a short-length catheter such as peripheral intravenous line having a magnetized distal end portion, etc.) to be placed in the blood vessel 1226, can be used to see if the blood vessel 1226 is able to accommodate the medical device.

### Methods for Following a Procedure for Placing a Medical Device in a Blood Vessel

Methods for following a procedure for placing a medical device (e.g., a needle, a short-length catheter such as peripheral intravenous line having a magnetized distal end portion, etc.) in a blood vessel (e.g., the blood vessel 1226) include a set of operations performed by executing instructions of a non-transitory CRM such as the memory 1123 by one or more processors (e.g., the processor 1122) of the ultrasound-imaging system 1110 that cause the ultrasound-imaging system 1110 to perform the set of operations, which include, in some embodiments, tracking a location of a tip of the medical device from a time of insertion at an insertion location, through a period of access in a targeted location of the blood vessel 1226, to a time of withdrawing the tip of the medical device from the insertion location. The tracking includes recording a duration of the procedure including intervals thereof (e.g., from the time of insertion through the period of access, from the period of access to the time of withdrawing the tip of the medical device, etc.), a depth of the blood vessel 1226, an angle of approach to the blood vessel 1226, an insertion angle at the targeted location of the blood vessel 1226, a number of readjustment passes during the procedure, or a combination thereof.

Following the procedure for placing the medical device in the blood vessel 1226 can include use of the ultrasound probe 1140 in a pulsed-wave Doppler imaging mode, the accuracy of which can be improved using a skin-adherable magnetic tag about an insertion location on a patient. With the magnetic tag in a known location (i.e., the insertion location) on the surface of the patient's skin, redshifts and blueshifts in ultrasound signals can be accurately calculated against reference magnetic signals of the magnetic tag for improving the accuracy of the Doppler image mode. For uniqueness among any other magnetic signals, the magnetic tag can be configured to switch on and off at a predetermined rate.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An ultrasound imaging system, comprising:
an ultrasound imaging probe; and
a console operably coupled to the ultrasound imaging probe, the console comprising one or more processors and memory including executable instructions that, when executed by the one or more processors, perform operations including:
determining a depth of a blood vessel with vessel depth-determination logic using data gathered by the ultrasound imaging probe above the blood vessel as input;
calculating whether a medical device is able to successfully access the blood vessel with medical device-accessibility logic using the depth of the blood vessel, an effective length of the medical device, an insertion location above the blood vessel, and an insertion angle of the medical device as inputs; and
displaying a visual indicator on a display over an ultrasound image, emitting an audio indicator from a speaker, or both to indicate whether the medical device will successfully access the blood vessel.

2. The ultrasound imaging system according to claim 1, wherein the ultrasound imaging probe includes a plurality of sensors, the operations further comprising determining the insertion location and the insertion angle of the medical device from sensor readings from the plurality of sensors.

3. The ultrasound imaging system according to claim 1, wherein the visual indicator is a target overlying the ultrasound image on the display, and wherein the target is configured to fade away, vanish, switch from one color to another color, or switch from one pattern to another pattern to indicate the medical device will not successfully access the blood vessel.

4. The ultrasound imaging system according to claim 1, wherein the visual indicator is an elongate graphical element overlying the ultrasound image that represents the effective length of the medical device.

5. An ultrasound imaging system, comprising:
an ultrasound imaging probe; and
a console operably coupled to the ultrasound imaging probe, the console comprising one or more processors and memory including executable instructions that, when executed by the one or more processors, perform operations including:
determining a depth of a blood vessel with vessel depth-determination logic using data gathered by the ultrasound imaging probe above the blood vessel as input;
calculating whether a minimum length of a medical device will be placed within the blood vessel with medical device-placement logic using the depth of the blood vessel, an effective length of the medical device, an insertion location above the blood vessel, and an angle of approach of the medical device as inputs; and
displaying a visual indicator on a display over an ultrasound image, emitting an audio indicator from a speaker, or both to indicate whether a potential placement of the medical device will result in the final placement of the sufficient length of the medical device within the blood vessel.

6. The ultrasound imaging system according to claim 5, wherein the ultrasound imaging probe includes a plurality of sensors, the operations further comprising determining the insertion location and the angle or approach of the medical device from sensor readings from the plurality of sensors.

7. The ultrasound imaging system according to claim 5, wherein the visual indicator is a target overlying the ultrasound image on the display, and wherein the target is configured to fade away, vanish, switch from one color to another color, or switch from one pattern to another pattern to indicate the medical device will not successfully access the blood vessel.

8. The ultrasound imaging system according to claim 5, wherein the minimum length of the medical device is user defined or set in accordance with a known minimum length provided by a manufacturer of the medical device.

9. The ultrasound imaging system according to claim 5, wherein the medical device is one of a needle and a short-length catheter.

10. The ultrasound imaging system according to claim 9, wherein the medical device is a short-length catheter, the set of operations further comprising:
estimating a distance a tip of the short-length catheter is from a tip of a needle in the blood vessel with tip-estimation logic as the short-length catheter is advanced over the needle; and
displaying an estimation of the distance on the display over the ultrasound image.
